(19) United States
(12) Patent Application Publication
(10) Pub. No.: US 2016/0346165 A1
(43) Pub. Date: Dec. 1, 2016

(54) CONTAINER FOR STORING AND/OR APPLYING A PHARMACEUTICAL SUBSTANCE AND METHOD OF ITS PRODUCTION

(71) Applicant: SCHOTT AG, Mainz (DE)

(72) Inventors: Bernd HOPPE, Ingelheim (DE); Klaus BAMBERG, Zuchwil (CH); Mustafa KÜÇÜK, Staad (CH)

(73) Assignee: SCHOTT AG, Mainz (DE)

(21) Appl. No.: 15/167,148

(22) Filed: May 27, 2016

(30) Foreign Application Priority Data
May 28, 2015 (DE) 10 2015 108 431.7

(51) Int. Cl. A61J 1/14; A61M 35/00; C03B 29/04; A61J 1/06

(52) U.S. Cl. CPC A61J 1/1468; A61J 1/065; A61M 35/003; C03B 29/04; A61J 2200/50; A61M 2207/00; A61M 2205/19; A61M 39/10

(57) ABSTRACT

A container for storing and/or applying a pharmaceutical substance is provided that includes a basic body made of glass and a first connecting body made of glass. The basic body has a substantially hollow cylindrical form and encloses a cavity. The basic body has a first end with a first opening. The first connecting body has a thin channel communicating with the first opening. The first connecting body is connected with the basic body in a first connection area. The first connection area has a first absorption zone that has a higher radiation absorption for electromagnetic waves in a predetermined wavelength range (λ) than portions of the basic body outside the first absorption zone (Z₁).

### CROSS REFERENCE TO RELATED APPLICATIONS

This application claims the benefit under 35 U.S.C. §119(a) of German Patent Application No. 10 2015 108 431.7 filed May 28, 2015, the entire contents of which are incorporated herein by reference

### BACKGROUND

### 1. Field of the Invention

The present invention relates to a container for storing and/or applying a pharmaceutical substance. A pharmaceutical substance is understood as being a substance such as a medicament, which is specifically used for treatment of the human or animal body. Pharmaceutical substances which may be stored in the container of the invention may comprise pasty, liquid, and gaseous substances and mixtures as well as dispersions and emulsions. Since glass is highly inert against a majority of commonly used pharmaceutical substances and has a high diffusion resistance, it is particularly suitable for storing pharmaceutical substances. Due to the high diffusion resistance permeation losses during storage are low, which is in particular an essential aspect for high-quality pharmaceutical substances.

### 2. Description of Related Art

Particularly in modern pharmaceutical active ingredients that are very expensive, highly effective and very sensitive, there is a growing tendency to use pre-filled syringes or carpules, wherein for the reasons mentioned above syringes made of glass are particularly suited. With pre-filled syringes it is no longer necessary to transfer the active ingredient from one container into another container. Rather, the pre-filled syringe is ready for use immediately after unpacking. Apart from saving time for the doctor or the nurse there is an additional advantage in that losses are avoided that frequently occur during the transfer from one container into the other. In addition, during the transfer there is a risk of infection or contamination of the substance and/or the syringe. The risk is considerably reduced with pre-filled syringes.

Syringes have a basic body, having a substantially hollow cylindrical form, which is why tubular glass is used for the basic body. In addition, syringes have relatively complicated geometries to connect cannulas or tubing for applying the pharmaceutical substances. As an example, a Luer-Lock connector is mentioned at this point, the manufacturing of which from glass involves considerable effort. The manufacturing of a Luer-Lock connector or other geometries directly from tubular glass, for which a multi-step hot-forming process with interlinked forming processes is performed, involves particularly considerable effort. All forming processes must be coordinated, as interlinking causes the forming processes to mutually influence one another as well as the forms obtained.

Alternatively, it is possible to connect a plurality of prefabricated connecting bodies made of glass, which already have the desired geometry, with the tubular glass. For example, the prefabricated connecting bodies may be connected by thermal joining methods, as a result of which an integral connection between the connecting bodies and the tubular glass is established. Due to the integral connection the syringe so produced has a high diffusion resistance, which is why it is as suitable for storing and/or applying pharmaceutical substances as the syringe directly manufactured from tubular glass. To this end, the tubular glass and the connecting bodies must be heated up to a temperature above the transformation point T_{G}, in which they cease to be dimensionally stable, so that also here manufacturing involves a considerable effort in order to manufacture the containers with the required accuracy.

In WO 96 024 73 A1 a light absorbing material is positioned between two glass plates which can thereby be bonded to each other. WO 2014/201315A1 shows a method in which a basic body made of glass is bonded with two glass layers in that the glass layers have a higher radiation absorption for electromagnetic waves than the basic body. In DE 10 2008 023 826 A1 a first member is connected with a second member by means of a connection solder, wherein the members as well as the connection solder consist of glass or glass ceramics, the connection solder having a higher radiation absorption than the two members.

US 2010/0280414 A1 shows a syringe, in which the connecting bodies are mechanically connected with the tubular glass without forming an integral connection. Such syringes, however, are not suitable for storing pharmaceutical substances, as they are either not sufficiently resistant to diffusion due to the mechanical connection, or the mechanical connection must be sealed with considerable effort, which is why sealing members can come into contact with the pharmaceutical substance. In both cases there remains a risk of bacteria and viruses, or other foreign substances, entering via the mechanical connection, which may lead to contamination of the pharmaceutical substance. Further, permeation losses via the sealing members may not be excluded, which is a great disadvantage given the usual expense of pharmaceutical substances.

Therefore, it is the object of the present invention to provide a container for storing and/or applying a pharmaceutical substance, which has a high diffusion resistance, keeps permeation losses within narrow limits, and is easily manufactured.

### SUMMARY

The container for storing and/or applying a pharmaceutical substance of the invention comprises a basic body made of glass, having a substantially hollow cylindrical form and enclosing a cavity, wherein the basic body has a first end with a first opening, and a first connecting body made of glass, wherein the first connecting body has a thin channel communicating with the first opening, the first connecting body is connected with the basic body in a first connection area, and the container has one or a plurality of first absorption zones within the first connection area, in which the container has a higher radiation absorption for electromagnetic waves in a predetermined wavelength range than the basic body outside the first absorption zone.

The first connecting body is either directly or indirectly connected with the first end of the basic body. According to the definition, the first connection area is to comprise the region of the contact surface of the basic body, via which the basic body either directly or indirectly contacts the first connecting body, but it may also slightly extend towards the center of the basic body, wherein the extension should be kept as low as is technically possible. On this basis, the first connection area is to comprise the whole connecting body. In this first connection area the absorption zone is arranged, in which the container has a higher radiation absorption for electromagnetic waves in a predetermined wavelength range than the basic body outside the first absorption zone. The first absorption zone is disposed within the first connection area such that the basic body can be connected either directly or indirectly with the connection bodies. Depending on the configuration of the container, the first absorption zone can be limited to the first end of the basic body. In this case, only the contact surface of the basic body, at which the basic body is directly or indirectly connected with the first connecting body, has a higher radiation absorption for electromagnetic waves. Alternatively, the first absorption zone can extend over the region of the first end of the basic body, including the contact surface. It is also conceivable that the first absorption zone wholly or partly extends over the first connecting body, wherein the region that interacts with the contact surface of the first connecting body is included. It is important that the first absorption zone does not extend over the whole basic body, but rather not at all, or only partly. Other constellations that are not mentioned here are also included.

Mercury vapor lamps that generate UV radiation, high-pressure xenon short-arc lamps that generate visible light rays, infrared radiation sources such as, for example, a Nd:YAG laser, a diode laser or a tungsten IR radiator, or a magnetron to create microwaves are mentioned as radiation sources in order to create the electromagnetic waves by way of example. The configuration of the container and, particularly, of the absorption zones is performed in consideration of the radiation sources used. In doing so, it is aimed to make a selection of the predetermined wavelength range that is as narrowly as is technically possible so that, preferably, only one wavelength is used, for which lasers are particularly suitable.

In the first absorption zone the container has a higher radiation absorption than the basic body outside the first absorption zone and, consequently, also outside the first connection area. It is thus possible to selectively heat the basic body and/or the connecting body in the first absorption zone locally more strongly under the action of electromagnetic waves than outside the first absorption zone, where the container has a lower radiation absorption. At least part of the basic body has a lower radiation absorption. In general, an increased radiation absorption may be brought about by increasing the absorption coefficient and/or by increasing the path length of the radiation in the first absorption zone. In doing so, the basic body and/or the connecting bodies is/are heated beyond the transformation point only in the region of the connecting point or the first contact surface, respectively, so that they are connected by an integral connection. The remaining region is heated less strongly so that this region is maintained dimensionally stable, causing no changes in dimension or form, which is a great advantage for accurate manufacturing.

In a further form of embodiment, a first joining body made of glass is arranged in the first connection area, via which the first connecting body is connected with the basic body. In this form of embodiment, a joining body which is arranged between the first connecting body and the basic body is used to connect the first connecting body with the basic body. The advantages and technical effects that may be such obtained correspond to those mentioned for the container described above. In doing so, the first absorption zone is not required to extend to the first joining body. It is sufficient if the first absorption zone extends over the region of the first end of the basic body, and wholly or partly over the first connecting body. In this case, the first joining body divides the first absorption zone into two parts, so that a plurality of first absorption zones is present. This form of embodiment is suitable, for example, for bridging differences in diameter between the basic body and the first connecting body.

In a further embodiment, the first absorption zone is limited to the first joining body. In other words, the first joining body exclusively forms the first connection area. Consequently, only the first joining body has an increased radiation absorption so that the basic body and the connecting body can remain completely unchanged, in order to connect them according to the method of the invention. In doing so, the first absorption zone is not required to fully extend over the first joining body. It is sufficient if the first joining body has an increased radiation absorption at its contact surfaces or the connecting points with the connecting body and the basic body. In this case, two first absorption zones are present. This allows the container of the invention to be manufactured in a particularly simple way and cost-efficiently.

In a further embodiment, the container comprises a second connecting body made of glass. In addition, the basic body has a second end with a second opening, wherein the second connecting body is connected with the basic body in a second connection area, and the container has one or a plurality of second absorption zones in the second connection area, in which the container has, at least in sections, a higher radiation absorption for electromagnetic waves in a predetermined wavelength range than the basic body outside the second absorption zone.

The advantages mentioned for the container having only the first connecting body also apply to this embodiment. In particular, this embodiment of the container of the invention is suitable for providing syringes for applying the pharmaceutical substances, for example, to the human or animal body, as the first connecting body may be embodied, for example, as a Luer-Lock connector and the second connecting body as a finger flange.

Luer-Lock connectors are widely used in laboratory, medical and pharmaceutical applications, for example, in order to connect tubing or cannulas to the first end. A Luer-Lock connector is a standardized component which substantially comprises an internal thread with a standardized, relatively large pitch, and a coaxially extending cone. Since the Luer-Lock connector must be manufactured according to standards, high demands are placed on its production regarding the accuracy, which may be realized according to the invention using the option of local heating at the point where the first connecting body has an increased radiation absorption for electromagnetic waves.

A piston may be inserted into the hollow cylindrical basic body via the second opening at the second end at which the finger flange is disposed. The piston is configured such that it seals the cavity against the respective second end such that no substance can escape at this end. Appropriate closures may be screwed into the Luer-Lock connector such that the container is also sealed at the first end in order to prevent leakage of the substance from the cavity. After the Luer-Lock connector is opened a cannula can be connected such that the substance may be conveniently applied, to which end the user can push the piston into the cavity with his thumb, while his fingers are supported on the finger flange. The finger flange can also have a “backstop” function such that the piston cannot be inadvertently removed from the cavity. According to the invention such pre-fillable syringes can be manufactured in a simple way and cost-efficiently.

In a further embodiment, a second joining body made of glass is arranged in the second connection area, via which the second connecting body is connected with the basic body. As already explained with regard to the first joining body, this embodiment is particularly suitable for bridging differences in diameter or form between the basic body and the second connecting body, which leads to a more flexible manufacturing process.

In doing so, the second absorption zone may be limited to the second joining body. It applies also here that the second connecting body may be more easily connected with the basic body, since it is possible to furnish only the second joining body with an increased radiation absorption for electromagnetic radiation. Again it is sufficient if the contact surfaces or the region of the connecting points have an increased radiation absorption, so that also a plurality of second absorption zones may be provided in the joining body.

In sum, it is therefore possible according to the invention to arrange the absorption zones as follows: In case the container does not comprise joining bodies, the absorption zones are arranged, on the basic body in the region of the contact surfaces, via which the basic body cooperates with the connecting body or bodies, and/or on the connecting body or bodies, at least in the region of the contact surfaces, via which the connecting body or bodies cooperate/s with the basic body.

In case the container has one or a plurality of joining bodies arranged between the basic body and the connecting body, the absorption zones are arranged, on the basic body in the region of the contact surfaces, via which the basic body cooperates with the joining body or bodies, on the connecting body or bodies in the region of the contact surfaces, via which the connecting body or bodies cooperate/s with the joining body or bodies, and/or on the joining body or bodies, at least in the region of the contact surfaces, via which the joining body or bodies cooperate/s with the basic body and the connecting body.

It is preferable for the basic body, the first connecting body, the second connecting body, the first joining body and/or the second joining body to consist of the same basic glass. The term basic glass, also referred to as glass type, refers to the fact that two glasses belong to the same basic glass if the composition of the main components and their concentrations as well as the chemical and physical properties are substantially the same, even though one glass may be doped with impurity atoms and the other one is not.

In particular, if the entire container or all bodies are manufactured from the same basic glass, the container of the invention has the same properties as a container that was directly manufactured from tubular glass. Modifications of the material are not required, which is a considerable advantage, particularly for the storage of pharmaceutical substances, as an approved basic glass may be used for the whole container, which clearly simplifies the approval of the container of the invention for storing pharmaceutical substances. In addition, the procurement of glass and storage of the basic glass, or of the basic bodies and the connecting bodies, respectively, are simplified, as it is not required to differentiate between different glass types. In sum, the container of the invention may be easily manufactured with a high tolerance and a dimensional stability from one and the same glass that is approved for the storage of pharmaceutical substances.

Further, it is preferable for the container in the first absorption zone or zones and/or the second absorption zone or zones consist of sintered glass. To this the first and/or the second connecting body and/or the first and/or the second joining body may for example consist of sintered glass. Here, for example, the connecting bodies or the joining bodies are manufactured from glass grains or glass powder by pressing and heating. In consequence, the connecting bodies or the joining bodies have a porosity that is different from the basic body. Reflection of the electromagnetic waves at the glass particles expands the path length which the electromagnetic waves must cover when passing the connecting body or the joining body produced from sintered glass in comparison with the basic body. In addition, diffusion is increased, which is why radiation absorption for accordingly selected electromagnetic waves is increased. Diffusion depends on the wavelength, so that the porosity and the diffusion surfaces (walls of enclosed air bubbles, particle boundary surfaces) must be adapted to the wavelength used. Porosity and diffusion surfaces can be particularly adjusted by way of the particle size of the glass grains or the glass powder.

In a further form of embodiment, the sintered glass comprises primary particles with a diameter D50 between 0.1 μm and 200 μm. Diameter D50 means that 50% of all primary particles have a diameter greater than the value indicated for D50. In this size range, on the one hand, it is possible to effectively increase radiation absorption of the preferably used electromagnetic radiation (visible light, infrared radiation); and the connecting bodies, the sintered glass of which has primary particles within this range of diameter, may be pressed particularly well. Herein, closed porosity is, preferably, from 0 to 50%. Closed porosity herein only considers self-contained cavities.

Typically syringes have a thin channel at the place where the cannula is connected. In the case of glass syringes, which are manufactured directly from tubular glass, this thin channel is manufactured using a tungsten pin which serves as a forming tool during the forming process. The heated glass is pressed onto the exterior surface of the tungsten pin in the region of the channel. After completion of the forming process the tungsten pin is removed from the syringe and the channel remains.

Without the use of the tungsten pin the thin channel may not be manufactured with the desired accuracy. In addition, there is a risk that the channel will be closed without the use of the tungsten pin during the forming process. Thus, the pin is made of tungsten, because it is able to withstand the high temperatures to which the glass has to be brought during the forming process in order to achieve the required viscosity without substantial chemical or mechanical changes. Here, however, it is disadvantageous that abrasion or evaporations occur when the tungsten pin is removed so that tungsten residues remain within the syringe which can migrate into the stored substance. This is particularly undesirable when pharmaceutical substances are stored in the syringe.

In contrast to this, the connecting body made of sintered glass may be manufactured with a thin channel without using tungsten pins, as forming is performed at room temperature, so that a decisive advantage in storing pharmaceutical substances in comparison with syringes made of tubular glass can be achieved. In addition, also connecting bodies having a more complex geometry may be manufactured more cost-efficiently by using sintered glass.

In a further embodiment the container is doped in the first absorption zone or zones and/or in the second absorption zone or zones for increasing the radiation absorption for electromagnetic waves. To this for example the first and/or the second connecting body and/or the basic body and/or the first and/or the second joining body may be doped to increase radiation absorption of electromagnetic waves. Here, impurity atoms are selectively introduced into the connecting bodies, the joining bodies and/or into the basic body, which increase the radiation coefficient and, consequently, radiation absorption. In doing so, the concentration of impurity atoms used approximately ranges from 0.1% to 5%. At this concentration radiation absorption is increased without changing the properties of the glass itself in a degree worth mentioning. Consequently, the doped glass has the same chemical and physical properties as the undoped glass with the exception of radiation absorption, so that doping has no negative effects on the manufacturing of the container and the storing of the pharmaceutical substances. Thus, it is the same basic glass. Consequently, a container is obtained which has the same properties in all places. Particularly advantageously, the first and/or the second connecting body may be doped with compounds of chromium, nickel, copper, iron, cobalt, rare earths (e.g., ytterbium, dysprosium) or with other elements, materials or compounds absorbing within the wavelength range of interest. When iron is used for doping, any iron oxide may be used, because a redox balance between iron-(II)-oxide and iron-(III)-oxide occurs in the glass. Combinations of the above mentioned compounds are also possible. When using sintered glass, doping may be performed by admixing the material which increases absorption of electromagnetic radiation in the desired concentration.

Some of the above mentioned materials cause a change in color in the doped glass during doping. For example, iron causes the doped glass to darken or to change its color to brown. Darkening or a change in color may be useful to mark the container, thus causing a visual differentiation. By means of the visual differentiation it can be ensured that a pharmaceutical substance is only filled into a container with a particular color mark. In addition, this may reduce the risk of confusion for doctors and nurses during the application.

When sintered glass is used, the materials used for doping may provoke a completely different change in color than in glass manufactured from doped bulk glass. Sintered bodies manufactured from glass powder or from doped bulk glass have a light grey, almost white appearance, so that the sintered body is very bright, which may also be used for marking purposes.

In a particular embodiment, the first and/or the second connecting body and/or the basic body and/or the first and/or the second joining body is/are formed of multi-phase sintered glass. Radiation absorption of the body formed of sintered glass may be precisely adjusted by the proportion of the phase which increases absorption of electromagnetic radiation. This is done by locally adjusting a clearly higher concentration of the material which increases absorption of electromagnetic radiation, for example, by admixing ceramic pigments. Thus, it is possible to dispense with doping, which is advantageous insofar that the concentrations of the material which increases absorption of electromagnetic radiation do not have to be adjusted too precisely.

In a further embodiment, the basic body may have a mating surface, and the first and/or the second connecting body may have a counter mating surface, at which the basic body is connected with the first and/or second connecting body, wherein the first and/or the second connecting body chemically and/or structurally differ/s from the basic body in the region of the counter mating surface. This also applies analogously to the joining bodies. There, the chemical composition and/or the structure is/are changed such that radiation absorption for electromagnetic waves is increased. Here, it is advantageous that radiation absorption is increased only in the regions of the mating surfaces and the counter mating surfaces, so that the other regions of the container are not heated in the joining process, so that they may soften and lose their form.

According to another embodiment the container is treated with a diffusion dye in the first absorption zone or zones and/or in the second absorption zone or zones. For this purpose, the first and/or the second connecting body may be treated with a diffusion dye in the region of the counter mating surface. Alternatively or cumulatively, the basic body may be treated with a diffusion dye in the region of the mating surfaces. This also applies analogously to the joining bodies. Diffusion dyes are, particularly, silver-containing substances, the components that cause a color effect of which enter adjacent and upper glass layers by diffusion during temperature treatment after application on the basic body and/or the connecting bodies, forming complex compounds with the glass. As a result, the upper glass layers change their color from yellow/dark yellow to red-brown, depending on the composition of the diffusion dyes, without significantly changing the mechanical and chemical properties. Radiation absorption for the correspondingly selected electromagnetic waves increases solely as a result of the coloring, in this case, for the visible and near infrared range. As treatment with diffusion dye is a relatively simple process, the effect according to the invention may be obtained without a significant additional effort.

Preferably, the glass, or the basic glass, respectively, is a borosilicate glass. Borosilicate glasses are characterized in that they have a particularly high inertia and resistance to chemicals, so that no undesired chemicals migrate from the borosilicate glass into the pharmaceutical substance. In addition, borosilicate glass can be easily sterilized, is gas tight and temperature-resistant.

Borosilicate glasses may comprise the following proportions in percent by weight:

SiO₂: 70% to 82%,

B₂O₃: 7% to 13%,

ΣNa₂O+K₂O: 4% to 8%,

Al₂O₃: 2% to 7%, and

ΣCaO+MgO: 0% to 5%.

Here it is worth mentioning that the number of components is relatively small, which allows a good prediction of the behavior with respect to the pharmaceutical substance. Borosilicate glass can be doped. However, as dopings are so small in proportion, particularly the chemical and mechanical properties will not be changed. The indicated proportions of borosilicate glass allow dopings to be performed.

Preferably, the first and/or the second joining body consist/s of sintered glass. The joining body may be manufactured from sintered glass very cost-efficiently, which, in addition, has an increased radiation absorption for the correspondingly selected electromagnetic waves solely due to the glass particles.

The first and/or the second joining body may differ chemically and/or structurally from the basic body and/or from the first or second connecting body. The chemical composition and/or the structure is/are changed at the desired locations such that radiation absorption for electromagnetic waves is increased. In doing so, the connecting bodies and the basic body may remain unchanged, so that the effort for manufacturing the container of the invention can be kept particularly low. For this purpose, the joining body may be treated with a diffusion dye, so that radiation absorption can be easily increased due to the effects described in more detail above.

The first and/or the second joining body can consist of, or comprise, a glass powder or a glass paste. Glass paste, herein, refers to glass powder bound with a liquid. In this embodiment, the joining body has similar properties as in the case where it consists of, or comprises, sintered glass. Due to higher radiation absorption the glass powder fuses, as a result of which the connecting bodies and the basic body are connected with one another. In case of glass paste the liquid evaporates when radiated, so that the glass powder is left.

In addition, the invention relates to a method for manufacturing a container for storing and/or applying a pharmaceutical substance, particularly according to any one of the exemplary embodiments described above, comprising the following steps:

Providing a basic body made of glass, having a substantially hollow cylindrical form and enclosing a cavity, wherein the basic body has a first end with a first opening, providing a first connecting body made of glass, comprising a thin channel, connecting the first connecting body at the basic body in a first connection area, in which the container has one or a plurality of first absorption zones, in which the container has a higher radiation absorption for electromagnetic waves in a predetermined wavelength range than the basic body outside the first connection area, wherein connecting is performed by irradiating at least the first connection area with electromagnetic waves in the predetermined wavelength range, as a result of which the container is heated more strongly by increased absorption of the electromagnetic waves in the first connection area than outside the first connection area, and the first connecting body is connected with the basic body such that the thin channel communicates with the first opening.

The advantages which may be obtained by the method of the invention correspond to those mentioned for the respective container. In summary, it is mentioned at this point that the process of manufacture may be simplified by pre-manufacturing the first connecting body accordingly, subsequently connecting it with the basic body in the manner mentioned above. Multi-step forming steps which must be exactly adapted to one another, are dispensed with, so that the container of the invention can be provided in a more cost-efficient and simple way than in the state of the art.

The method of the invention is further developed by the following steps: arranging a first joining body made of glass in the first connection area between the basic body and the first connecting body, and connecting the first joining body with the first connecting body and the basic body by irradiating at least the first absorption zone with electromagnetic waves in the predetermined wavelength range.

Besides the advantages described for the above mentioned exemplary embodiments, the container of the invention can be manufactured by this method in a particularly simple way and cost-efficiently, because only the joining body must have an increased radiation absorption for electromagnetic waves. Both the connecting body and the basic body may remain unchanged.

The method of the invention is further developed by the following steps: providing the basic body made of glass, having a second end with a second opening, providing a second connecting body, connecting the second connecting body at the basic body in a second connection area, in which the container has a second absorption zone, in which the container has a higher radiation absorption for electromagnetic waves than the basic body outside the second absorption zone, wherein connecting is performed by irradiating at least the second absorption zone with electromagnetic waves in the predetermined wavelength range, as a result of which the container is heated more strongly by increased absorption of the electromagnetic waves in the second absorption zone than outside the second absorption zone.

The container manufactured in this manner is particularly suitable for use as a pre-filled syringe for applying the pharmaceutical substance.

The method of the invention is further developed by the following steps: arranging a second joining body made of glass in the second connection area between the basic body and the second connecting body, and connecting the second joining body with the second connecting body and the basic body by irradiating at least the second absorption zone with electromagnetic waves.

A container manufactured by this method is particularly suitable for bridging differences in diameter and form between the basic body and the connecting body.

The method of the invention, wherein the basic body has a mating surface at the first end or in the region of the first end and/or at the second end or in the region of the second end, and the first and/or the second connecting body has a counter mating surface, at which the basic body is connected with the first and/or the second connecting body, is further developed by the following step: roughening the mating surface and/or the counter mating surface before performing the steps of arranging and irradiating.

In doing so, a similar effect as in sintered glass is obtained such that also here radiation absorption for accordingly selected electromagnetic waves is increased without the need to additionally introduce doping. In doing so, the advantage is obtained that no traces are left on the finished container, which refer to the roughened mating surfaces and/or counter mating surfaces, providing a particularly homogeneous container.

Process steps described for the manufacture of the container without separate joining bodies may also be applied for the manufacture of the container with a joining body.

The connecting bodies can be manufactured from a glass drop by means of pressing, from tube sections or glass plates by means of hot forming, from glass powder by means of laser sintering (rapid prototyping method), or by means of a ceramic 3D print with subsequent sintering. If the connecting bodies are manufactured from glass drops, tube sections or glass plates, the increase of radiation absorption for electromagnetic waves is preferably obtained by doping, roughening or using diffusion dyes. If the connecting bodies are manufactured by sintering it is possible to dispense with doping, roughening, or the use of diffusion dyes, as radiation absorption may already be sufficiently increased by diffusion at the particles of the sintered glass.

In doing so, it is particularly preferable for the basic body, the first connecting body, the second connecting body, the first joining body and/or the second joining body to consist of the same basic glass. In particular, this may simplify storage, as only one basic glass must be purchased and stored.

Further the container may consist of sintered glass in the first absorption zone or zones and/or the second absorption zone or zones. By using sintered glass the radiation absorption for electromagnetic waves may be increased in an easy way. It is not necessary to take further measures for increasing the radiation absorption for electromagnetic waves. Additionally by using sintered glass more complex geometries may be produced which would not be possible with normal glass.

Further, the invention relates to the use of a container according to any one of the above described exemplary embodiments for storing and/or applying a pharmaceutical substance.

### BRIEF DESCRIPTION OF THE DRAWINGS

The present invention is explained in detail using preferred exemplary embodiments with reference to the attached figures.

FIG. 1 shows a first exemplary embodiment of a container of the invention in an unconnected state,

FIG. 2 shows a second exemplary embodiment of the container of the invention in an unconnected state,

FIG. 3 shows a basic illustration of a method for manufacturing the container according to the first exemplary embodiment, and

FIG. 4 shows a basic illustration of a method for manufacturing the container according to the second exemplary embodiment.

### DETAILED DESCRIPTION

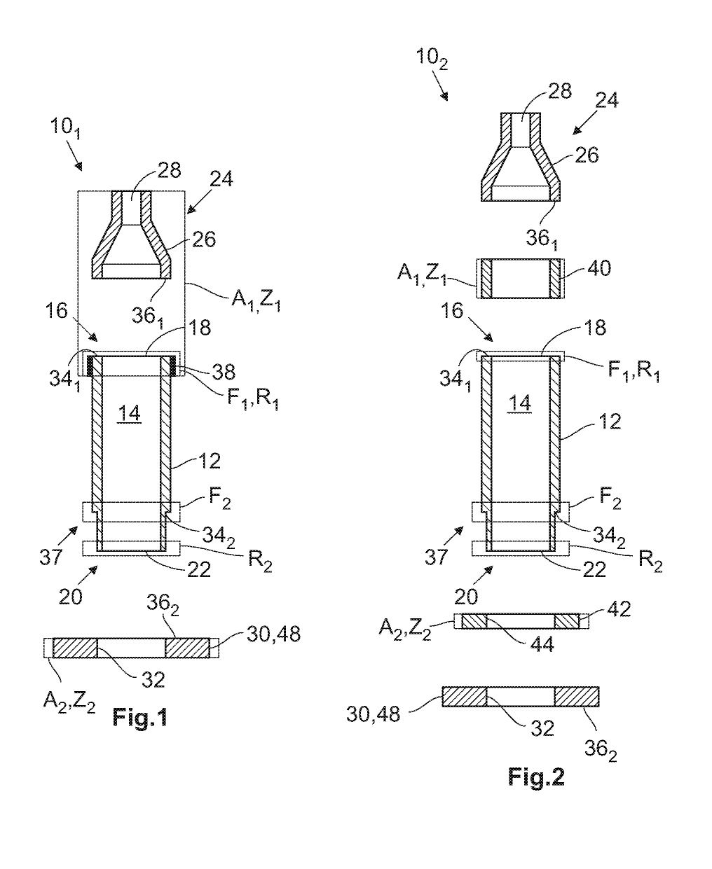

FIG. 1 shows a first exemplary embodiment of a container of the invention 10₁ in an unconnected state. The container 10₁ comprises a basic body 12, having a substantially hollow cylindrical form and enclosing a cavity 14. Thus, the basic body 12 has a first end 16, which encloses a first opening 18, and a second end 20, which encloses a second opening 22.

Further, the container of the invention 10₁ comprises a first connecting body 24, which is connected with the basic body 12 in a first connection area A₁. In the present case, the first connection area A₁ is defined such that it encloses a region of the first end R₁ of the basic body 12 and extends from said portion over the whole connecting body 24. The first connecting body 24 has a cone-shaped section 26 and a thin channel 28. The first connecting body 24 may have connecting geometries that are not illustrated in more detail, for example, a Luer-Lock connector for connecting a cannula or a tubing.

Further, the container 10₁ has a second connecting body 30, which is configured approximately annularly and has a passage opening 32, which in its diameter approximately corresponds to the outer diameter of the basic body 12 in a step 37. In this case, a second connection area A₂ only extends to the second connecting body 30.

In addition, the basic body 12 has a first mating surface 34₁ and a second mating surface 34₂, each, respectively, cooperating with a first counter mating surface 36₁ of the first connecting body 24 and a second counter mating surface 36₂ of the second connecting body 30, as will be explained in more detail below. In the illustrated example, the second mating surface 34₂ is arranged in the step 37 of the basic body 12.

In the region F₁ of the first mating surface 34₁ the basic body 12 differs from the remaining region such that the basic body 12 has an increased radiation absorption for electromagnetic waves in the region F₁ of the first mating surface 34₁. For this purpose, the basic body 12 may be roughened at the first mating surface 34₁. Alternatively, the basic body 12 is roughened at the first mating surface 34₁ and has been treated with a diffusion dye 38 in the region R₁ of the first end 16, which in this case coincides with the region F₁ of the first mating surface 34₁. In a further alternative, the basic body 12 has been treated with a diffusion dye 38 in the region R₁ of the first end 16, which in this case coincides with the region F₁ of the first mating surface 34₁, without the mating surface 34₁ having been roughened. The two last alternatives are illustrated in FIG. 1.

The respective regions F₁, R₁ are understood as being regions, comprising in each case the first end 16 or the first mating surface 34₁, respectively, but additionally as being a region of the basic body 12 that is selectable in size.

In the illustrated example, the first connecting body 24 has an overall higher radiation absorption for electromagnetic waves, for example, because it is manufactured from a sintered glass. Thus, it does not need to contain any additional doping, but may already be more radiation-absorbent solely due to the increased diffusion. Consequently, the container 10₁ has a first absorption zone Z₁ in the first connection area A₁, which in this case comprises the first connecting body 24 and the region of the first end R₁ of the basic body, thus coinciding with the first connection area A₁. Consequently, an absorption zone Z₁ is understood to comprise all regions within the connection area A₁, which have an increased absorption for a predetermined wavelength range λ.

However, it is equally possible to manufacture only a portion of the first connecting body 24, comprising the counter mating surface 36₁, from sintered glass, so that only this portion has an increased radiation absorption. In this case, the first absorption zone Z₁ extends over the region of the first end R₁ of the basic body 12, and only partly over the connecting body 24, so that the absorption zone Z₁ does not coincide with the connection area A₁, but is only part of it.

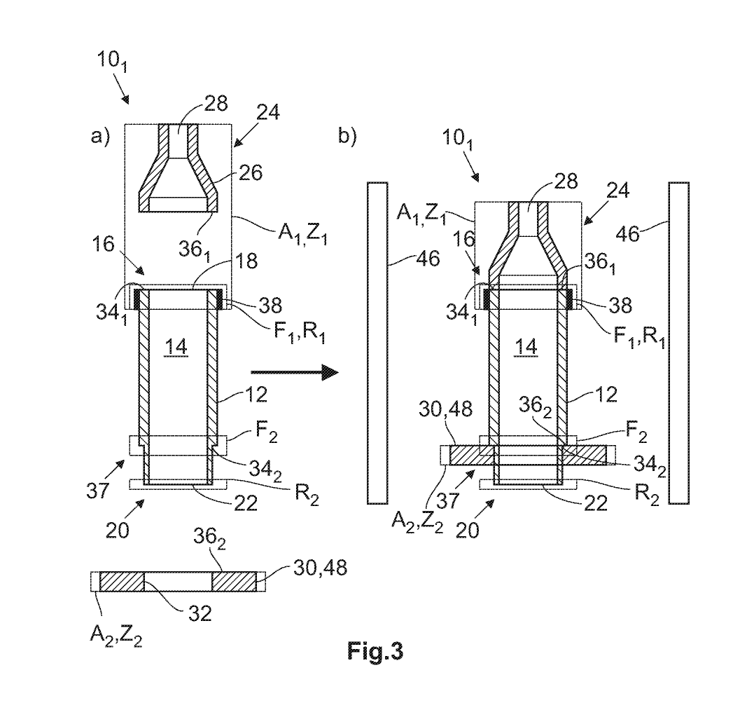

In this illustrated example, the second connecting body 30 can also be manufactured from sintered glass. The region R₂ of the second end 20 may, depending on the configuration of the basic body 12, comprise region F₂ of the second mating surface 34₂, wherein the two regions F₂, R₂ do not need to be equally sized. As the second connecting body 30 consists of sintered glass and has a higher radiation absorption for electromagnetic waves as a result, it is not necessary to specifically configure the basic body 12 in the region of the second mating surface 34₂ or in the region R₂ of the second end 20. In this case, the container 10₁ has a second connection area A₂, which only extends over the second connecting body 30, but does not include the region of the second end R₂ (cf. FIG. 3b)). Correspondingly, a second absorption zone Z₂ extends over the second connecting body 30 and coincides with the second connection area A₂.

Alternatively, in the region F₂ of the second mating surface 34₂, or in the region R₂ of the second end 20, the basic body 12 may be configured in the same manner as in the region F₁ of the first mating surface 34₁ or in the region R₁ of the first end 16. In this case, the second connection area A₂ and also the second absorption zone Z₂ still comprise the region F₂, but not the region of the second end R₂.

Both the first connecting body 24 and the second connecting body 30 as well as the basic body 12 consist of the same basic glass, particularly, of a borosilicate glass.

FIG. 2 shows a second exemplary embodiment of the container of the invention 10₂ also in an unconnected state, which substantially corresponds to the first exemplary embodiment 10₁. In addition, however, the container 10₂ of the second exemplary embodiment has a first joining body 40, which is arranged at the first end 16 between the basic body 12 and the first connecting body 24. Further, the container 10₂ comprises a second joining body 42, which is configured approximately annularly, having a passage opening 44, the diameter of which corresponds to the external diameter of the basic body 12. In the illustrated example, only the first and the second joining body 40, 42 have an increased radiation absorption, whereas the first and the second connecting body 24, 30 and the basic body 12 have not undergone any treatment which has the consequence of an increase in radiation absorption. Consequently, the container 10₂ has a first connection area A₁, extending over the first joining body 40 and coinciding with the first absorption zone Z₁. Further, the container 10₂ has a second connection area A₂, extending over the second joining body 42 and coinciding with the second absorption zone Z₂.

FIG. 3 illustrates a method for manufacturing the container 10₁ according to the first exemplary embodiment by means of schematic sketches. On the basis of the unconnected state illustrated in FIG. 3a), the first end 16 of the connecting body 24 is disposed on the basic body 12 such that the first mating surface 34₁ contacts the first counter mating surface 36₁. The second connecting body 30 is slid over the second end 20 onto the basic body 12 until the second counter mating surface 36₂ bears on the second mating surface 34₂ in the step 37. Subsequently, the container 10₁ is irradiated with electromagnetic waves of a predetermined wavelength λ in an aggregate which is not illustrated in more detail, for which an radiation source 46 is provided (see FIG. 3b)). Depending on the radiation source 46 used, a wavelength range A may be used herein. As a result of irradiation, the container 10₁ is heated more strongly in the first absorption zone Z₁ than outside the first absorption zone Z₁. In the illustrated example, the first connecting body 24 is heated more strongly, as it is manufactured from sintered glass. Further, the basic body 12 is heated more strongly in the region R₁ of the first end 16, as it is coated with the diffusion dye 38 there. The connecting body 24 and the region R₁ of the first end together form the first connection area A₁, which coincides with the first absorption zone Z₁. The diffusion dye 38 may be configured such that silver compounds are formed in the near-surface layers of the basic body 12. Also if an increased radiation absorption is present only in the near-surface layers, and these layers are initially heated up due to irradiation, the basic body 12 will heat up by thermal conduction more strongly in the whole region R₁ of the first end 16 than in the remaining region.

On the second end 20, only the second connecting body 30 is heated more strongly, as it is also manufactured from sintered glass. The basic body 12 has not been specially treated with respect to an increased radiation absorption, so that it is not heated more strongly. Therefore, the second connection area A₁ coincides with the second absorption zone Z₂.

The radiation source 46 is operated such that the first and the second connecting bodies 24, 30 and the region R₁ of the first end 16 are heated to a temperature above the transformation point T_{G}, particularly, above the softening point EW. The other regions are only heated to temperatures below the softening point EW but may be in the range of the transformation point T_{G}. Consequently, the viscosity of the two connecting bodies 24, 30 is reduced overall by irradiation, and of the basic body 12 it is reduced in the region R₁ of the first end 16 by irradiation, and additionally of the basic body 12 by thermal conduction within the region F₂ of the second mating surface 34₂, forming an integral connection between the basic body 12 and the connecting bodies 24, 30 as a result. A hermetically sealed connection is obtained during cooling. As the other regions of the basic body 12 are heated to temperatures below the softening point EW, in particular, below or in the range of the transformation point T_{G} as a result of irradiation, it will not deform, remaining dimensionally stable. Thermal post-treatment may be performed to remove tension in the container 10₁. However, as the container 10₁ is not only heated in the region of the connection point between the basic body 12 and the connecting bodies 24, 30, tension is limited. In addition, the radiation source 46 is not required to be specifically adapted, which simplifies the configuration of the aggregates.

Additionally, the container 10₁ can be pre-heated before and/or during treatment with the radiation source 46 in order to keep differences in temperature between the individual components 12, 24, 30 as low as possible, so that high thermal tensions are avoided which can destroy the components or the resulting connection 10₁.

In a connected state, the second connecting body 30 acts as a finger flange 48, so that the now completed container 10₁ can be used as a pre-fillable syringe for storing and applying a pharmaceutical substance.

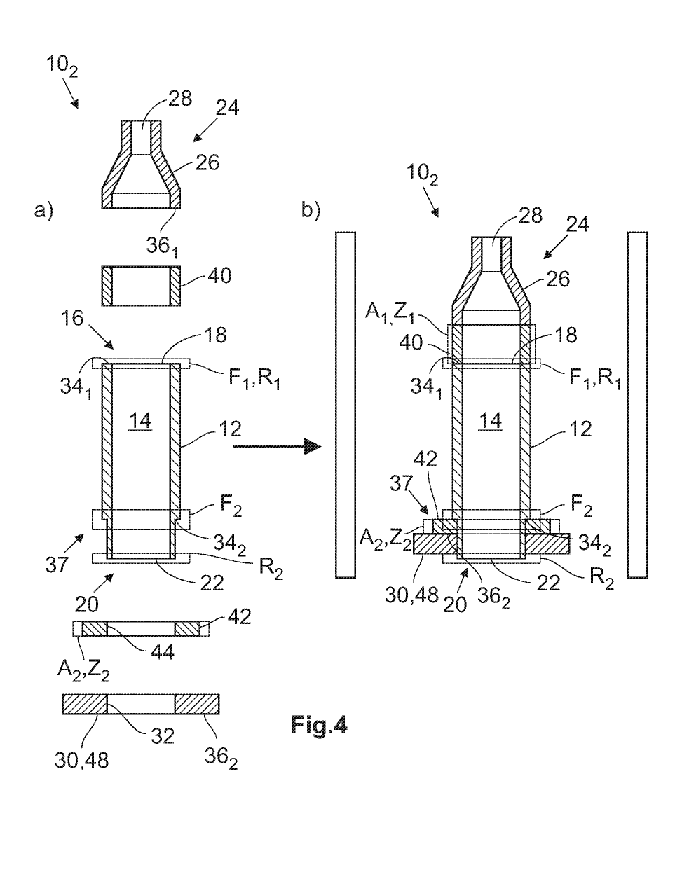

FIG. 4 represents a basic illustration of a method for manufacturing the container 10₂ according to the second exemplary embodiment. The container 10₁ of the second exemplary embodiment is substantially manufactured in the same way as the container 10₁ of the first exemplary embodiment with the exception that the first or second joining body 40, 42 is placed between the basic body 12 and the first and the second connecting bodies 24, 30. In the illustrated example, only the two joining bodies 40, 42 are to have an increased radiation absorption, so that these are heated to a temperature above the transformation point T_{G}, in particular, above the softening point EW, melt, and, consequently, form an integral connection with the basic body 12 and the first connecting body 24 or the second connecting body 30, respectively. In doing so, the basic body 12 and the first and second connecting body 24, 30 are heated to a temperature below the softening point EW, but within the range of the transformation point T_{G}, so that they do not deform. A hermetically sealed connection is obtained during cooling. Thermal post-treatment may be performed to remove tension in the container 10₁. In a connected state, the second connecting body 30 acts as a finger flange 48, so that the completed container 10₂ can be used as a pre-fillable syringe for storing and applying a pharmaceutical substance.

Preferred radiation sources for the creation of electromagnetic waves each comprise one or a plurality of UV radiation sources, for example, mercury vapor lamps and/or radiation sources which emit in the visible range, for example xenon short-arc high-pressure lamps and/or infrared radiation sources, in particular infrared radiation sources emitting short-wave infrared radiation, for example, Nd:YAG lasers, diode lasers, or tungsten IR radiators, and/or microwave radiation sources, for example, magnetrons. Short-wave infrared radiation (sw IR radiation) generated by tungsten halogen IR radiators with a color temperature of 1500 to 3500K has proved to be particularly suitable. In the case of this heating technology, heating is not solely determined by the temperature of the aggregate, but substantially by the IR radiation of the heating elements and the absorption behavior of the body to be heated.

### First Exemplary Embodiment (Tungsten Halogen IR Radiator)

Starting point is an arrangement as shown in FIG. 4, consisting of a basic body 12 made of a borosilicate tubular glass of a total length of 45 mm and having an external diameter of 8 mm and two connecting bodies 24, 30 made of sintered glass, also of the same borosilicate glass, both doped with 5% Fe₂O₃. The basic body 12 and the connecting bodies 24, 30 are arranged as shown and passed through a continuous furnace at a speed of from 1 cm/s to 10 cm/s. At the level of the joining bodies 40, 42 irradiation from tungsten halogen IR radiators as a radiation source 46 with a color temperature of from 1500 to 3000 K is directed at the container 10₂ from the outside. The infrared radiation performance is set such that the connecting bodies 24, 30 fuse within 1 to 60 sec to hermetically bond and seal them to the basic body 12. The whole container 10₂ is heated by a conventional additional heater with 500 W electrical power, or an infrared heater, or another suitable heating device to several hundred ° C. during infrared irradiation such that no inadmissibly high tensions may occur within the basic body 12 or within the connecting bodies 24, 30 during local infrared irradiation. After successful fusion a further thermal post-treatment is excluded in order to remove remaining tensions from the now completed container 10₂.

### Second Exemplary Embodiment (Laser)

Starting point is an arrangement as shown in FIG. 4, consisting of a basic body 12 made of a borosilicate glass tubing with a total length of 45 mm and an external diameter of 8 mm as well as two connecting bodies 24, 30 made of sintered glass, also made of the same borosilicate glass, which are doped with 5% Fe₂O₃. The basic body 12 and the connecting bodies 24, 30 are fixed perpendicularly on a rotation plate and rotated with a rotational speed of from 1 to 120 rpm. On the level of the joining bodies 40, 42 irradiation is radially directed from the outside with a laser beam of a wavelength of between 900 to 1500 nm to the connecting bodies 24, 30. In doing so, a suitable device serves to widen the laser beam, so that a laser line of approximately 4 mm in length is generated. Laser performance is set such that the joining bodies 40, 42 fuse within 1 to 60 sec to hermetically bond and seal the basic body 12 to the connecting bodies 24, 30. The whole container 10₂ is heated by a conventional additional heater with 500 W electrical power, or an infrared heater, or another suitable heating device to several hundred ° C. during infrared irradiation such that no inadmissibly high tensions may occur within the basic body 12 or within the connecting bodies during local infrared irradiation. After successful fusion a further thermal post-treatment is excluded in order to remove remaining tensions from the now completed container 10₂.

### Third Exemplary Embodiment (Microwave Resonator)

Starting point is an arrangement as shown in FIG. 4, consisting of a basic body 12 made of a borosilicate glass tubing with a total length of 45 mm and an external diameter of 8 mm as well as two connecting bodies 24, 30 made of sintered glass, also made of the same borosilicate glass, which are filled with 1 to 90% Fe. The basic body 12 and the connecting bodies 24, 30 are fixed perpendicularly on a rotation plate and rotated with a rotational speed of from 1 to 120 rpm in a cylindrical microwave resonator with an internal diameter of 30 mm, wherein microwave radiation with a frequency of 0.9 to 30 GHz is coupled into the microwave resonator by means of a hollow microwave conductor. The performance of the microwave resonator may be adjusted by pulsing or other suitable control measures such that the joining bodies 40, 42 fuse within 1-60 sec to hermetically bond and seal the basic body 12 to the connecting bodies 24, 30. The whole container 10₂ is heated by a conventional additional heater with 500 W electrical power, or an infrared heater, or another suitable heating device to several hundred ° C. during infrared irradiation such that no inadmissibly high tensions may occur within the basic body 12 or within the connecting bodies 24, 30 during local infrared irradiation. After successful fusion a further thermal post-treatment is excluded in order to remove remaining tensions from the now completed container 10₂.

### LIST OF REFERENCE SIGNS

10, 10₁, 10₂ Container
12 Basic body
14 Cavity
16 First end
18 First opening
20 Second end
22 Second opening
24 First connecting body
26 Cone-shaped section
28 Thin channel
30 Second connecting body
32 Passage opening
34, 34₁, 34₂ Mating surface
36, 36₁, 36₂ Counter mating surface
37 Step
38 Diffusion dye
40 First joining body
42 Second joining body
44 Passage opening
46 Radiation source
48 Finger flange
A₁ First connection area
A₂ Second connection area
F₁ Region of the first mating surface
F₂ Region of the second mating surface
R₁ Region of the first end
R₂ Region of the second end
Z₁ First absorption zone
Z₂ Second absorption zone

What is claimed is:

1. A container for storing and/or applying a pharmaceutical substance, comprising:
a basic body made of glass, the basic body having a substantially hollow cylindrical form that encloses a cavity, the basic body has a first end with a first opening, and
a first connecting body made of glass, the first connecting body having a thin channel,
the first connecting body is connected with the first end of the basic body in a first connection area so that the thin channel communicates with the first opening, the first connection area having a first absorption zone with a higher radiation absorption for electromagnetic waves in a first predetermined wavelength range than portions of the basic body outside the first absorption zone.

2. The container according to claim 1, wherein the first absorption zone comprises sintered glass.

3. The container according to claim 1, further comprising a first joining body made of glass is arranged in the first connection area, the first joining body connecting the first connecting body with the first end of the basic body.

4. The container according to claim 3, wherein the first absorption zone is limited to the first joining body.

5. The container according to claim 3, further comprising a second connecting body made of glass, wherein the basic body has a second end with a second opening, the second connecting body is connected with the second end of the basic body in a second connection area, the second connection area has a second absorption zone with a higher radiation absorption for electromagnetic waves in a second predetermined wavelength range than portions of the basic body outside the second absorption zone.

6. The container according to claim 5, further comprising a second joining body made of glass arranged in the second connection area, the second joining body connecting the second connecting body with the second end of the basic body.

7. The container according to claim 6, wherein the second absorption zone is limited to the second joining body.

8. The container according to claim 7, wherein one or more of the basic body, the first connecting body, the second connecting body, the first joining body, and the second joining body comprise a common glass.

9. The container according to claim 5, wherein the first and second predetermined wavelengths are one wavelength.

10. The container according to claim 5, wherein the first and/or the second absorption zones comprise sintered glass.

11. The container according to claim 10, wherein the sintered glass comprises primary particles with a diameter D50 of between 0.1 μm and 200 μm.

12. The container according to claim 10, wherein the first and/or the second absorption zones comprise doping that increases radiation absorption for electromagnetic waves.

13. The container according to claim 5, wherein the basic body has a mating surface and one of the first and second connecting bodies has a counter mating surface, the mating surface is connected with the counter mating surface in a region, wherein the one of the first and second connecting bodies is chemically and/or structurally different from the basic body in the region.

14. The container according to claim 13, further comprising a diffusion dye in the region.

15. The container according to claim 14, wherein the diffusion dye is on one or more of the basic body and the one of the first and second connecting bodies.

16. The container according to claim 1, wherein the glass is borosilicate glass.

17. The container according to claim 16, wherein the borosilicate glass comprises, in percent by weight:
SiO₂: 70% to 82%,
B₂O₃: 7% to 13%,
ΣNa₂O+K₂O: 4% to 8%,
Al₂O₃: 2% to 7%, and
ΣCaO+MgO: 0% to 5%.

18. A method for manufacturing a container for storing and/or applying a pharmaceutical substance, comprising:
providing a basic body made of glass, the basic body having a substantially hollow cylindrical form that encloses a cavity, the basic body has a first end with a first opening,
providing a first connecting body made of glass, the first connecting body having a thin channel,
connecting the first connecting body with the first end of the basic body in a first connection area so that the thin channel communicates with the first opening, the first connection area having a first absorption zone with a higher radiation absorption for electromagnetic waves in a first predetermined wavelength range than portions of the basic body outside the first absorption zone,
wherein the step of connecting comprises irradiating at least the first absorption zone with electromagnetic waves in the first wavelength range so that the first absorption zone is more strongly heated than the portions outside the first absorption zone due to increased absorption of the electromagnetic waves.

19. The method according to claim 18, further comprising arranging a first joining body made of glass in the first connection area between the first end of the basic body and the first connecting body, wherein the step of connecting comprises connecting the first joining body with the first connecting body and the first end of the basic body by irradiating at least the first absorption zone with the electromagnetic waves.

20. The method according to claim 19, wherein the step of providing the basic body further comprises providing the basic body with a second end having a second opening, the method further comprising:
providing a second connecting body made of glass, and
connecting the second connecting body at the second end of the basic body in a second connection area, the second connection area having a second absorption zone with a higher radiation absorption for electromagnetic waves in a second predetermined wavelength range than portions of the basic body outside the second absorption zone,
wherein the step of connecting comprises irradiating at least the second absorption zone with electromagnetic waves in the second wavelength range so that the second absorption zone is more strongly heated than the portions outside the second absorption zone due to increased absorption of the electromagnetic waves.

21. The method according to claim 20, further comprising:
arranging a second joining body made of glass in the second connection area between the second end of the basic body and the second connecting body, wherein the step of connecting comprises connecting the second joining body with the second connecting body and the second end of the basic body by irradiating at least the second absorption zone with the electromagnetic waves.

22. The method according to claim 21, wherein the providing steps comprise providing one or more of the basic body, the first connecting body, the second connecting body, the first joining body, and the second joining body of a common glass.
